(19) United States
(12) Patent Application Publication
(10) Pub. No.: US 2025/0295326 A1
(43) Pub. Date: Sep. 25, 2025

(54) Systems and Methods for Emulating Electrical Impedance Tomography Using Audio Transducers

(71) Applicant: Meera IYER, Fremont, CA (US)

(72) Inventor: Meera IYER, Fremont, CA (US)

(21) Appl. No.: 19/080,885

(22) Filed: Mar. 16, 2025

Related U.S. Application Data
(63) Continuation-in-part of application No. 18/616,086, filed on Mar. 25, 2024.
(60) Provisional application No. 63/644,730, filed on May 9, 2024.

(51) Int. Cl. A61B 5/08; A61B 5/00; A61B 7/04

(52) U.S. Cl. CPC A61B 5/0816; A61B 5/6802; A61B 5/7225; A61B 5/7267; A61B 7/04

(57) ABSTRACT

Systems and techniques for creating clinically useful emulations of EIT scans by using audio sensors to capture sounds from the thoracic cavity, particularly but not solely lung sounds, where a neural network-based Audio Encoder is, in an embodiment, trained by simultaneously performing both an EIT scan and an audio recording of a cohort of test patients. The EIT scans are passed through an image encoder/decoder pair, each of which can also be neural network-based, and the image encoder creates an embedding representative of the EIT scan. The frequency characteristics of the audio signals are captured as an intermediate representation and supplied to the audio encoder which is trained to map an embedding of the converted audio to the embedding of the EIT scan from the image encoder. At run time audio of a patient is processed through the frequency conversion and supplied to the now-trained Audio Encoder to generate an embedding. The embedding is supplied to the image decoder which produces the EIT emulation for review by a clinician.

### RELATED APPLICATIONS

This application is a conversion of U.S. Patent Application Ser. No. 63/644,730 filed May 9, 2024, and further is a continuation-in-part of U.S. patent application Ser. No. 18/616,086 filed Mar. 25, 2024, which in turn is a conversion of U.S. Patent Application Ser. No. 63/491,957 filed Mar. 24, 2023. The present application claims the benefit of each of the foregoing, all of which are incorporated herein by reference.

### FIELD OF THE INVENTION

The present invention relates generally to medical imaging and more particularly to methods and systems for generating clinically useful emulations of EIT images using audio signals captured at one or more positions on the thoracic cavity of a human subject which signals are then manipulated by artificial intelligence techniques to yield emulations of EIT images.

### BACKGROUND OF THE INVENTION

Electrical Impedance Tomography (EIT) is a relatively recent, non-invasive, and radiation-free imaging technique for assessing lung function and, in some instances, heart function. EIT allows physicians to measure regional lung ventilation and to quantify lung collapse, tidal recruitment and lung overdistension, and may also help to detect pneumothorax and perfusion. EIT also offers the possibility of continuously monitoring cardiac stroke volume, among other uses. Unfortunately, most commercial EIT systems are expensive and bulky and, perhaps even more significantly, require trained personnel to affix various electrodes to the patient. To perform EIT measurements, trained medical personal place electrodes around the thorax in a transverse plane, usually in the 4^{th} to 5^{th} intercostal spaces. While it is unquestioned that EIT offers numerous benefits, roughly two-thirds of the world's population, especially those located remotely or in dangerous areas such as combat zones, do not have access even to basic radiology services such as X-rays, let alone EIT.

EIT is generally a low resolution (typically 32×32 images), high frequency (33 frames/second) imaging modality. In at least some implementations, sixteen electrodes are placed around the chest. Current is applied between consecutive pairs, and voltages are measured at other positions. For each frame, numerous voltage measurements are taken, for example 208. The goal is to estimate the electrical conductivity of the lungs at each instant using the 208 measurements. The electrical conductivity equation to be solved is the Laplace Equation, which is a partial differential equation:

$$∇ \cdot ( σ ( x , y ) ∇ u ( x , y ) ) = 0$$

g(x,y) is the conductivity
u(x,y) is the electrical potential.

The equation can be solved by well-known algorithms such as the GREIT algorithm, implemented in the popular EIDORS package for EIT. Solving the equation provides the conductivity image within the lungs, which provides the physician an idea of how air is moving through the image at a given instant in time. However, while the benefits of EIT imaging can be readily appreciated, the challenges noted above remain.

On the other hand, stethoscopes, and particularly the sensors therein which detect audio signals, are readily accessible, comparatively cheap, portable, and easy to use even without significant training. Chest auscultation, a process where one or more sensors such as those found in stethoscopes are placed at different locations on a patient's chest and back, is another way to assess indirectly the functioning of the lungs, heart and other organs. Such “stethoscope” audio provides information about air moving through the lungs, blood moving through the heart, and other fluids moving in or through other organs. AI methods have been used in other contexts to generate images from audio.

Many implementations of AI methods involve developing embeddings as representations of the original data stream. Embeddings are compressed representations of data (images, sound, text, etc) that can be trained by a deep neural network for tasks such as classification or reconstruction of images or other forms of data where that reconstructed output closely matches the original image or other data. The creation of an embedding can be thought of as an encoding, while the reconstruction can be thought of as a decoding. As used herein, an example of an embedding, i.e., smaller dimensional mathematical entity, might be a vector of real numbers of fixed dimension, for example 64, 128, etc.

While generative AI methods and neural networks have been used to produce solutions to many technical challenges, it has not previously been known to produce clinically useful emulations of EIT images from audio signals obtained via stethoscope-type sensors from various positions on the chest. The availability of such systems and methods will allow physicians to assess lung and organ function more quickly and easily, especially for remote patients who do not have easy access to trained medical personnel, in turn enabling earlier detection of disease or other injury and generally improving the quality of in-person and virtual patient care. It can thus be appreciated that there has been a long-felt need for systems and methods by which audio signals from the thoracic cavity can be processed to provide clinically useful emulations of EIT images.

### SUMMARY OF THE INVENTION

The present invention provides methods and systems by which a deep neural network can be trained using, in an embodiment, actual EIT images and audio signals from the same patient. The EIT images created with conventional EIT systems are then encoded to an embedding to train an Image Encoder. The encoded images are then used to train an Image Decoder, the output of which is essentially congruent with the original EIT image. For each patient, the breath sounds from the audio sensors are digitally recorded and then converted to an intermediate representation that captures the frequency characteristics of the audio signals. If more than one audio sensor is used, the recorded audio signals can be processed separately or in the aggregate. As discussed hereinafter, the audio sensor or sensors can be one or more digital stethoscopes, analog stethoscopes or other similar audio transducers as are well known in the art. An Audio Encoder, sometimes hereinafter referred to as a Stethoscope Sounds Encoder (SSE) is then trained to map the intermediate representations of the audio signals to the embedding of corresponding EIT image created by the Image Encoder. The result is that embeddings from the audio signals now match closely to the embeddings derived from the corresponding EIT image.

Once the neural networks that form the Audio Encoder and the Image Decoder are sufficiently trained, the system can execute the run time phase. During run time, breath sounds from a new patient are recorded from the one or more audio sensors and are processed to create corresponding intermediate representations. During capture of patient breath sounds, the audio sensors are typically positioned at auscultation locations on a patient's thoracic cavity. As noted above, the audio sensors can be configured as part of a digital or other stethoscope, or can be transducers integrated into a garment such as a vest that can be worn tightly around the torso.

The intermediate representations are then passed through the now-trained Audio Encoder to obtain embeddings representative of the recorded audio signals. The resulting embeddings are then passed through the now-trained Image Decoder. The output of the trained Image Decoder is an image that emulates the image that a conventional EIT system would have created, but without the equipment, time, cost, and trained personnel required to obtain an EIT image.

Depending upon the embodiment and in some cases the specific implementation, the Image Encoder, Image Decoder, and Audio Encoder can be any of a variety of known structures, for example a Variational Auto Encoder (“VAE”) which is a neural network-based encoder/decoder pair whose encoder is trained to produce an embedding from each input EIT image from a training dataset of images, and whose decoder is trained to reconstruct the original EIT image back from the embedding. Alternative embodiments, and in some cases specific implementations, may instead choose to use a conditional Generative Adversarial Network (cGAN), Principal Component Analysis (PCA), a Linear Auto Encoder, and others. In the event a cGAN is used, one option is to use a standard image encoder such as ResNet101 to create the image embedding and then use a cGAN to regenerate an image close to the original. This invention is not intended to be limited to any specific choice for encoder+decoder pair.

The audio sensors mentioned herein can be of any suitable type, either digital or analog, and may for example include conventional stethoscopes, wearable stethoscopes, wearable garments, and so on, where the key requirements are that the audio sensors are positioned appropriately to record breath sounds and other sounds from the thoracic cavity. If analog sensors are used, an A/D conversion is used to enable digital recording. More generally, if different types of transducers with different characteristics are used, calibration steps can be added if appropriate to the embodiment. In one approach, both, the digital stethoscope sounds for training data and the transducer sounds from run-time may be passed through the same filtering process to retain similar frequencies, and same background noise cancelling process so that the sound data fed to the audio encoder during training and testing are largely of similar character. An more elaborate alternative approach can also be used where digital stethoscope sounds and the transducer sounds are both captured simultaneously or in quick succession from a number of human subjects. Breath cycles are detected from each set. A spectrogram is created from the breath sounds from both sources. A variational autoencoder (VAE) is trained to regenerate the spectrogram from the digital stethoscope spectrogram. An encoder is trained to produce the same embedding as the VAE from the microphone sounds.

It is one object of the invention to provide a system and method for providing an image which is a clinically useful emulation of an EIT scan through the use of recorded audio signals.

It is a further object of the invention to provide a audio capture system and method suitable for use in generating emulations of EIT scans.

Another object of the invention is to provide methods and techniques for converting audio signals into intermediate representations from which embeddings representative of the audio signals can be encoded into embeddings.

It is yet another object of the invention to provide an encoder/decoder pair trained to convert representations of recorded audio into images depicting the human thoracic cavity.

These and other objects of the invention will be better appreciated from the following Detailed Description of the Invention taken together with the appended figures described below.

### THE FIGURES

FIG. 1 illustrates in block diagram form the elements of an embodiment of an aspect of the invention.

FIG. 2 illustrates in flow diagram form the training phase of an embodiment of an aspect of the invention.

FIG. 3 illustrates in flow diagram form the run time phase of an embodiment of an aspect of the invention.

FIG. 4 illustrates a garment with audio sensors integrated therein in accordance with an embodiment of an aspect of the invention.

FIG. 5 illustrates a computer system for executing instructions in accordance with an embodiment of the method and system of the present invention.

### DETAILED DESCRIPTION OF THE INVENTION

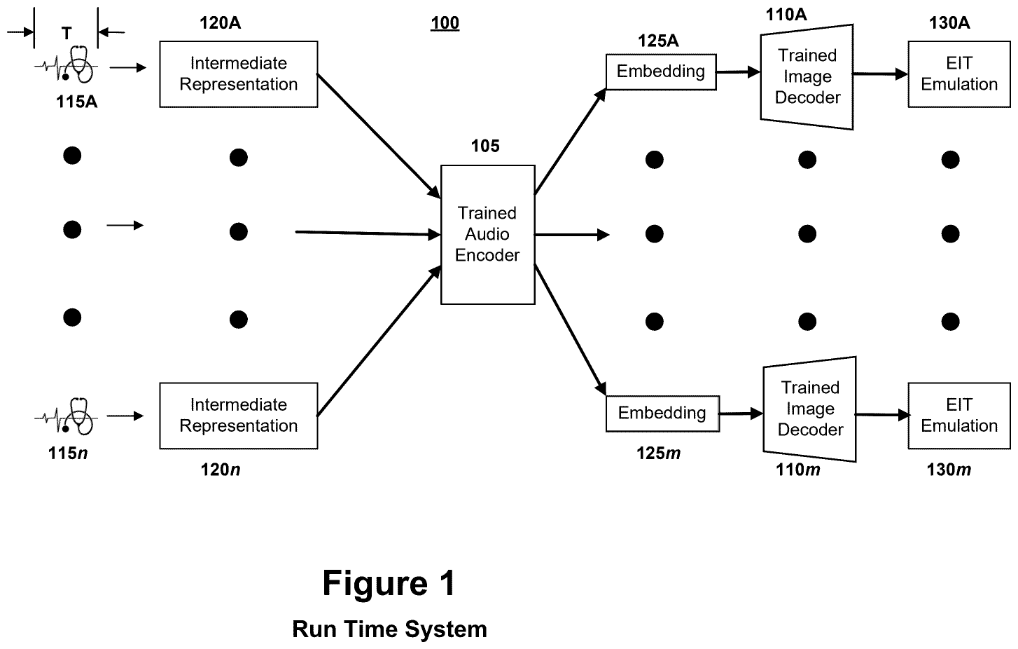

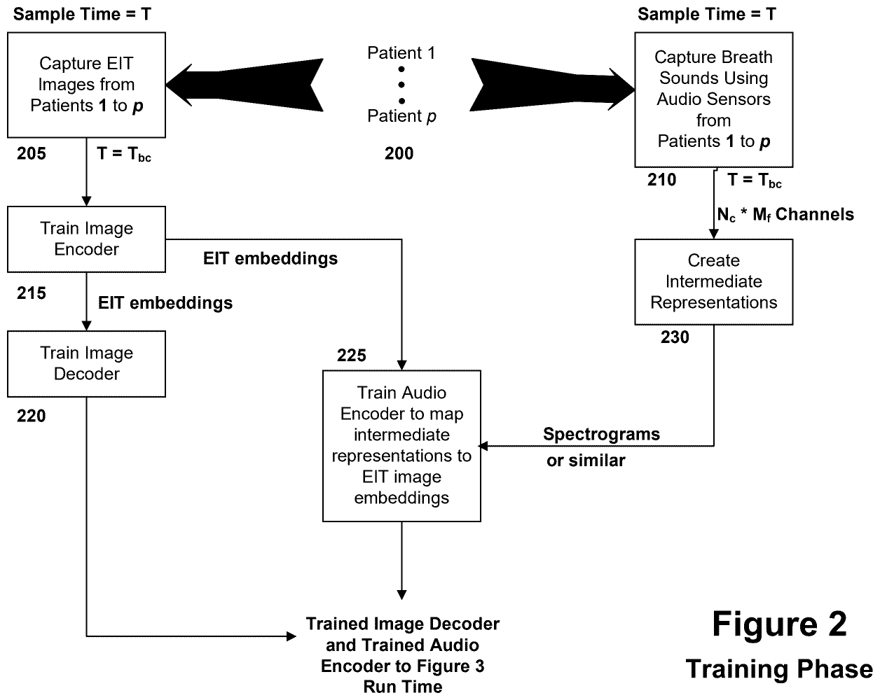

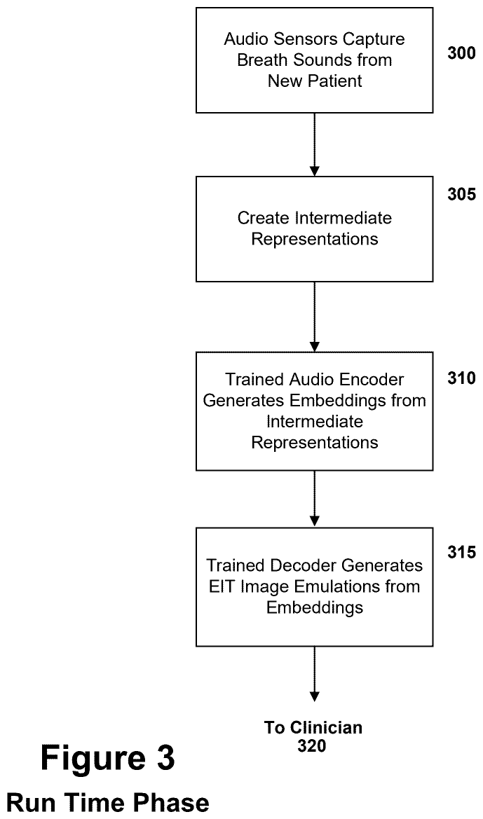

Referring first to FIGS. 1-3, in FIG. 1 a system for performing the method of an embodiment of the present invention, indicated generally at 100, can be appreciated, including one or more trained audio encoders 105 and one or more trained image decoders 110. The training processes for the audio encoder and image decoder are shown in FIG. 2 and discussed in greater detail hereinafter. The process of the run time process for the system of FIG. 1 is shown in FIG. 3. Still referring to FIG. 1, one or more audio sensors 115A-115n each provide an audio signal lasting a duration T, where in at least some embodiments T is long enough to capture at least one breath cycle. In some embodiments T can be longer than a single breath cycle and the system performs an analysis such that the effective T will match the duration of one or more breath cycles. For convenience, a breath cycle as used herein is the time duration between two consecutive significant events of the same type such as inhalation/inspiration or exhalation/expiration. A given patient's breath cycle may be detected by the use of a breathing sounds segmentation method known in the art by providing as input at least one of the audio signals captured by a properly placed stethoscope or other audio sensor, or by any other convenient means or methods.

Each of the audio signals 115A-115n, which typically comprise digital data, is then converted into an intermediate image representation 120A-120n, that captures the frequency characteristics of each audio signal. For example, such a conversion can be achieved by calculating from the audio data a spectrogram, a mel spectrogram, an MFCC [Mel-Frequency Cepstral Coefficients], or any other suitable method for capturing the frequency characteristics of the audio data, for example a fixed-size audio embedding generated by a pre-trained audio encoder. For embodiments with multiple sensors positioned around the thoracic cavity with each constituting a channel C, a total number of channels N_{c} is processed by the logic of the intermediate representations 120A-120n. In addition, for improved clarity, in some embodiments each of the channels of data C can be filtered through one or more digital filters F. In such embodiments, where the number of filters is M_{F}, the total number of channels of data is N_{C}*M_{F}.

The various channels of data are then passed through an audio encoder or SSE 105, which is in at least some embodiments a deep neural network that has been trained as discussed below with particular reference to FIG. 2. It will be appreciated by those skilled in the art that, because the audio signals 115A-115n are provided either by a digital sensor or by an analog sensor whose output has been converted to digital format, the data presented to the audio encoder 105 will be a stream of samples of audio data. In some embodiments, a sampling rate of 4 KHz may be used although the sampling rate can vary over a wide range, both lower and higher. For example, a 48 KHz sampling rate is used for many types of audio signals.

The output of the audio encoder for each channel is an embedding 125A-125m, that is representative of the frequency characteristics of the audio signals 115A-115n. The embeddings may comprise a multiple of the n audio signals given the possibility of a plurality of filters as noted above. Each such embedding is then supplied to a trained image decoder 110A-110m. The image decoders 110A-110m are trained as discussed in connection with FIG. 2, below, and are a deep neural network configured to decode the embeddings into digital images that emulate the results of an EIT scan of the patient whose audio signals were recorded by the present invention. The result is a set of images 130A-130m. It will be appreciated that additional processing may combine one or more of the images 130A-130m to provide the clinician either a clearer final image or a compression of the data that permits a faster analysis.

With reference particularly to FIG. 2, an embodiment of the training phase of the system of FIG. 1 can be better understood. First, a training dataset must be developed. For one embodiment of the training dataset, a patient cohort 200 of p patients is gathered. For each such patient, an audio recording of breath sounds is made concurrently with performing an EIT scan on that patient. The result is that a set of p EIT scans 205 is matched to a set of p audio recordings 210. The size of the patient cohort can vary depending upon the clinical objectives, but in many implementations it may be helpful to have a cohort that represents a range of symptoms for the medical conditions being monitored, including patients with a diverse range of symptoms characteristic of healthy, early stage disease, and so on up through advanced cases of a given medical condition.

A unit of time T is selected as the time period for which both the audio is captured and for which EIT images are to be generated. As discussed above, the particular time T will typically be long enough to record at least one breath cycle for every patient in the cohort 200. However, because the breath rates of humans can vary significantly, from a few breaths per minutes to more than one per second, a fixed time T may cause excessive and possibly unnecessary data to be captured. Thus, in some embodiments an analysis may be performed and the duration of both the EIT scan and the audio recording may be selected to be a time T_{bc} comprising one or more complete breath cycles for each individual patient in the cohort 200. For example, in an embodiment T_{bc} could be 7.76 seconds which, at a typical EIT frequency Fₑ of 33 images per second, would result in N=T*Fₑ=256 EIT images for the duration of time T_{bc}.

In an embodiment, the EIT measurements are converted into P images by performing a Laplace transform on the EIT data in a manner well known in the art. The EIDORS toolkit may be used to create the P images by providing the EIT measurements to the software. In an embodiment, each EIT image may be of size 32×32 pixels, although larger sizes are created in other embodiments, and the present invention is not limited to a particular size of EIT image.

The image data from 205 is the used to train an Image Encoder shown at step 215 which typically is part of an encoder/decoder pair such as a Variational Auto Encoder (VAE). In an embodiment, the VAE's encoder may be a convolutional neural network (CNN) with a fully connected layer as the last layer. In an embodiment the convolutional neural network may comprise three convolutional layers and the ReLU activation function. The first layer may be of size 16 channels×16 width×16 height. The second convolutional layer may be of size 32×8×8. The third convolutional layer may be of size 64×4×4. In an alternative embodiment the VAE's decoder may have a fully connected layer, followed by three convolutional transpose layers. In some embodiments, the fully connected layer may be of size 64. In another alternative embodiment, the fully connected layer may be followed by a convolutional transpose layer of size 32×8×8, a convolutional transpose layer of size 16×16×16, and a final convolutional transpose layer of size 3×32×32, corresponding to the red, green, and blue channels of the final image output.

By processing the P images from step 205, the Image Encoder of step 215 is trained to reduce each EIT image into an embedding of a fixed size. In an embodiment, the embedding may be of size D=64. The embeddings created by the Image Encoder at step 215 are then provided at step 220 to an Image Decoder which comprises the other half of the Image Encoder/Decoder pair noted above. The function of the Image Decoder is essentially to reverse the embedding process of step 215 by reconstituting, or decoding, the embeddings of step 215 into a close approximation of the original image. It will be appreciated that the Image Decoder will typically have the same architecture as the Image Encoder.

In addition to providing the EIT embeddings to step 220, the output of step 215 is provided to step 225, where an Audio Encoder is trained to cause the intermediate representations of the one or more recorded breath sounds of step 210 to be converted into D-dimensional embeddings that match the EIT embeddings of step 215 from the same breath cycle. More specifically, and as discussed in connection with FIG. 1, the recorded audio signals from step 210 are passed to step 230, where intermediate representations of the audio signals are created like those in FIG. 1 at 120A-n but here are used for training. As with FIG. 1, an audio signal from at least one position on the chest and back from one or more humans with a duration of, preferably, at least one breath cycle, is converted into an intermediate representation, such as a mel spectrogram.

The output of the intermediate representations step comprises N_{C}*M_{F} channels of spectrogram image data or similar representations. That data is then supplied to the Audio Encoder training step shown at 225. The model which comprises the Audio Encoder is trained to take as input the N_{C}*M_{F} channels of spectrogram image data and produce as output a sequence of (T_{bc}*Fₑ) embeddings where Fₑ is the number of scans per second performed by the EIT device, as discussed above. One embodiment of the Audio Encoder model is a deep convolutional neural network, whose weights are trained in order to minimize a loss function that encourages the output sequence of (T_{bc}*Fₑ) image embeddings to be as close as possible to the (T_{bc}*Fₑ) embeddings from the real EIT images corresponding to the same time interval from the same patient. In one embodiment, the Audio encoder/SSE may be a convolutional neural network. In an embodiment the audio encoder/SSE may have six blocks of a convolutional layer, a batch norm layer, and a max pool layer, followed by N fully connected layers, which produce N outputs of embeddings of size D each. In an embodiment, the loss may be an L1 or Mean Absolute Error loss between the D-dimensional embeddings produced by the Image Encoder at step 215 (FIG. 2) and the D-dimensional embeddings produced by the Audio Encoder as described in connection with step 225 (FIG. 2). In an embodiment, the audio encoder or SSE is trained on datasets collected from at least one human subject. In other embodiments, the loss terms can include L1 loss plus other terms that encourage the objective. For example, one such additional term is called “perceptual loss” where the image is reconstructed from the prior learned embedding, and a feature extractor (typically another neural network) is applied to that reconstructed image to reduce the L1 loss between that and the feature vector of the original. Training is considered complete only when the loss is below a predetermined threshold, which can vary with the implementation context.

The results of the image encoding step 215, image decoding step 220, and audio encoding step 225 are to create Trained Audio Decoder 105 and Trained Image Decoders 110A-m which are then utilized during Run Time, as discussed above in connection with FIG. 1 and discussed in greater detail below with reference to FIG. 3, below.

In the event that matched pairs of EIT images and audio recordings are not available for training, an EIT image of a patient known to have a specific type and severity of a given disease may be matched with recorded breath sounds of a different patient known to have approximately the same severity of that disease. In this regard, a disease classification, a clinician's notes, or similar medical information can be used to train the audio encoder and map the resulting embeddings to EIT-based embeddings. A Large Language Model [LLM] or similar can be used to capture such text information and process it to yield the desired embeddings. Thresholding can be used to ensure adequate similarity between the text embeddings of the notes associated with the EIT image of a first patient and text embeddings of the notes associated with the audio recording of a second patient. It will be appreciated by those skilled in the art that while the foregoing assumes the audio encoder is being trained at least in part by such alternative information, the converse approach is also possible, i.e. using a clinician's notes, etc., to validate an EIT image.

At run time, as shown in FIG. 3, audio signals from a new patient, typically not a member of the cohort 200 but taken in the same number and from the same positions on the thoracic cavity as those recorded from the patients of cohort 200, are captured in any convenient means and manner as shown at 300. Those one or more signals are then converted to intermediate representations in step 305 using the same filters and conversion techniques as used during step 230 of the training phase shown in FIG. 2 and shown in FIG. 1 at 120A-120n. The chosen intermediate representations are then passed through the trained Audio Encoder 105 discussed above, which produces (T_{bc}*Fₑ) embeddings. Next, each of the embeddings is passed through the Trained Image Decoders 110A-110m which constructs (T_{bc}*Fₑ) emulations of EIT images. Those images are then either provided to a clinician or submitted for downstream processing.

As noted above, in some embodiments it may be desirable to perform the emulation using one or more complete breath cycles. This requires detecting the beginning and end of at least one breath cycle, where the breath cycle comprises an inhale/inspiration portion together with an exhale/expiration portion. The beginning and end of the cycle can be detected by, first, converting the audio recordings of lung sounds into spectrograms or equivalent as discussed above. Once that conversion is made, the beginning and ending of breath cycles can be thought of as a “bounding box” where the left of the box is the beginning time and right the ending time. The well-known FasterRCNN object detection system can be then be used for the bounding box, or boundary, detection task. FasterRCNN involves two CNNs: a Region Proposal Network (RPN) for identifying potential breathing phases and their locations, and a classification network for processing these proposals. Adjustments to the FasterRCNN include using ResNet101 architecture pre-trained on ImageNet and adapting the output layer to classify three classes—background, inspiration, and expiration. The FasterRCNN method can be trained on subjects whose breath cycles are manually annotated. At run time, a subject's sound data is passed through the FasterRCNN networks to produce bounding boxes that indicate the start and end of breath cycles.

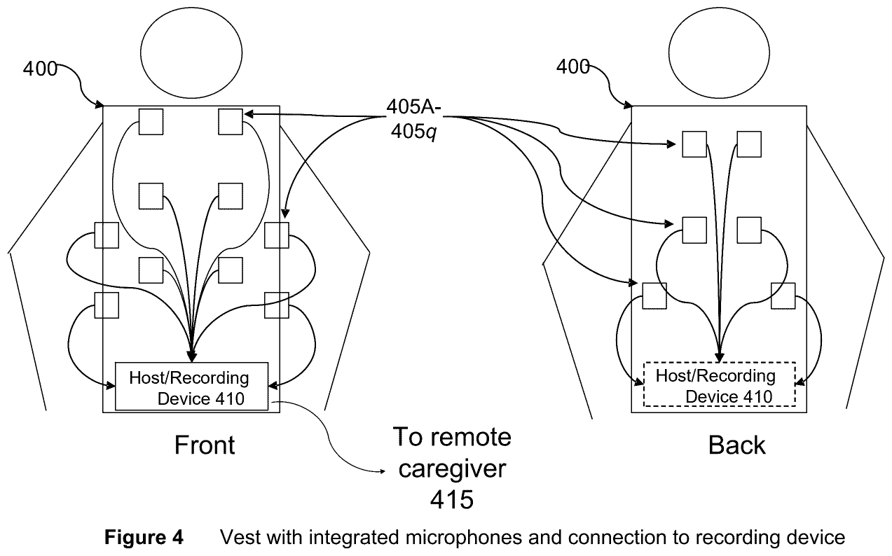

The present invention may be combined with a wearable stethoscope that captures audio from multiple locations of the thorax in a wearable garment such as the vest 400 shown in FIG. 4, where a plurality of stethoscope-type sensors 405A-405q are integrated into the vest and connected wirelessly to a host 410, potentially for transmission through the internet to a remote caregiver 415. The captured multichannel audio data can be input into the presented system for the generation of images that emulate EIT scans. The sensors 405A-405q may also sense signals from the infrasonic and ultrasonic bands of the audio spectrum in a manner which can in some embodiments further enhance the accuracy of the emulated EIT images.

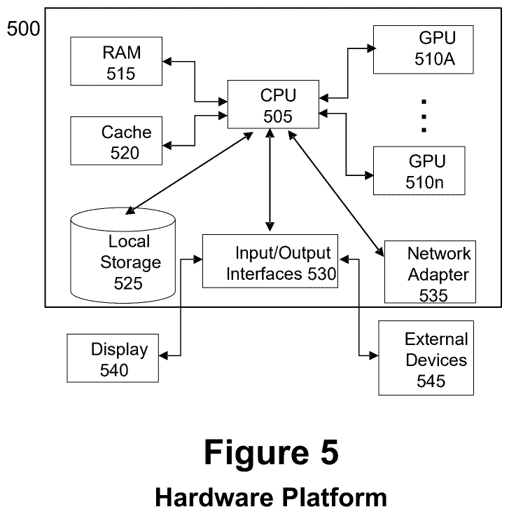

Referring next to FIG. 5, an embodiment of a hardware platform 500 suitable for executing each of the functions described herein can be appreciated. A CPU 505 communicates bidirectionally with one or more optional GPU's 510A-510n as well as RAM 515, cache 520 and local storage 525. The CPU also communicates bidirectionally with I/O interfaces 530 and network adapter 535. The I/O interfaces in turn communicate bidirectionally with a display 540 and other external devices 545 such as keyboard, mouse, and so on.

In some embodiments described herein, plural instances may implement components, operations, or structures described as a single instance and vice versa. Likewise, individual operations of one or more embodiments may be illustrated and described collectively where, alternatively, one or more of the individual operations may be performed concurrently, and the operations may be performed in an order different than that illustrated. Structures and functionalities presented as separate components in example configurations may be implemented as a combined structure or single component. Similarly, structures and functionalities presented as single components or structures may be implemented as a one or more structures or components. These and other variations, modifications, additions, and improvements fall within the scope of the subject matter herein.

Embodiments described herein as including components, modules, mechanisms, functionalities, steps, operations, or logic may comprise either software modules (e.g., code embodied on a machine-readable medium or in a transmission signal) or hardware modules. As just one example, an embodiment of the present invention can comprise a non-transitory computer readable storage medium comprising stored instructions for training an encoder used to develop emulations of emulations of EIT images from recorded breath sounds of at least a portion of the thoracic cavity, the instructions when executed causing at least one processor and data storage in communication therewith to: call from a data store at least one EIT image from each of a plurality of patients; call from a data store at least one set of recorded breath sounds from each of the same plurality of patients, where a set of recorded breath sounds corresponds to an EIT image for a given patient; encode at least some of the EIT images to generate an associated EIT embedding; generate an intermediate representation of at least some of the set of the recorded breath sounds corresponding to the encoded EIT images; and map the EIT embedding and the corresponding intermediate representation to generate a breath sounds embedding such that the loss for the resulting EIT embedding and the resulting breath sounds embedding is below a predetermined threshold.

A related aspect of the invention comprises a non-transitory computer readable storage medium comprising stored instructions for developing from recorded breath sounds emulations of EIT images of at least a portion of the thoracic cavity, the instructions when executed causing at least one processor and data storage in communication therewith to: call a set of recorded breath sounds of a patient; generate an intermediate representation of the set of recorded breath sounds; call an audio encoder trained to map intermediate representations to EIT image embeddings; generate in the trained audio encoder an embedding of the intermediate representation of the set of recorded breath sounds; call an image decoder trained to generate EIT images from EIT image embeddings, and in the trained image decoder, decode the embedding of the intermediate representation to generate an emulated EIT image.

A hardware module comprises a tangible unit configured or arranged to perform the requisite operations. In example embodiments, one or more computer systems (e.g., a standalone, client or server computer system, co-located or remote from one another) or one or more hardware modules of a computer system (e.g., a processor or a group of processors) may be configured either by software (e.g., an application or application portion) or as a hardware module that operates to perform certain steps or operations as described herein.

In various embodiments, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within one or more general-purpose processors or other programmable processors) that is temporarily configured by software to perform certain operations. It will be appreciated that the implementation of a hardware module in a particular configuration may be driven by cost and time considerations.

Embodiments in which one or more hardware modules are temporarily configured (e.g., programmed), each of the hardware modules need not be configured or instantiated at any one instance in time. For example, where the hardware modules comprise a general-purpose processor and/or a graphics processor configured using software, one or more such processors may be configured as respective different hardware modules at different times. Software may accordingly configure a processor, for example, to constitute a particular hardware module at one instance of time and to constitute a different hardware module at a different instance of time.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g., the Internet) and via one or more appropriate interfaces (e.g., application program interfaces (APIs).) The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Some portions of this specification are presented in terms of equations, algorithms or symbolic representations of operations on data stored as bits or binary digital signals within a machine memory (e.g., a computer memory). These equations, algorithms or symbolic representations are examples of techniques used by those of ordinary skill in the machine learning and data processing arts to convey the substance of their work to others skilled in the art. As used herein, an “algorithm” is a self-consistent sequence of operations or similar processing leading to a desired result. In this context, algorithms and operations involve manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are to be understood merely as convenient labels associated with appropriate physical quantities.

Unless specifically stated otherwise, terms such as “processing,” “computing,” “calculating,” “determining,” “presenting,” “displaying,” “generating”, “emulating” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The phrase “in an embodiment” used in various places in the specification do not necessarily all refer to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present). Having fully described a preferred embodiment of the invention and various alternatives, those skilled in the art will recognize, given the teachings herein, that numerous further alternatives and equivalents exist which do not depart from the invention. Thus, while particular embodiments and implementations have been illustrated and described, it is to be understood that the invention is not limited to the precise embodiments, structures and configurations disclosed herein but is to be limited only by the appended claims.

I claim:

1. A method for training an audio encoder to emulate encoded EIT images comprising the steps of
providing from a data store at least one EIT image from each of a plurality of patients,
providing from a data store at least one set of recorded breath sounds from each of the same plurality of patients, where a set of recorded breath sounds corresponds to an EIT image for a given patient,
in one or more processors, encoding at least some of the EIT images to generate an associated EIT embedding,
generating in the one or more processors an intermediate representation of at least some of the set of the recorded breath sounds corresponding to the encoded EIT images, and
mapping, in the one or more processors, the EIT embedding and the corresponding intermediate representation to generate a breath sounds embedding such that the loss for the resulting EIT embedding and the resulting breath sounds embedding is below a predetermined threshold.

2. A method for emulating EIT images using breath sounds comprising the steps of
providing from a first data store a set of recorded breath sounds of a patient,
generating in one or more processors an intermediate representation of the set of recorded breath sounds,
providing from a second data store an audio encoder trained to map intermediate representations to EIT image embeddings,
generating, in the one or more processors executing the trained audio encoder, an embedding of the intermediate representation of the set of recorded breath sounds,
providing from a third data store an image decoder trained to generate EIT images from EIT image embeddings, and
in the trained image decoder executing in the one or more processors, decoding the embedding of the intermediate representation to generate an emulated EIT image.

3. A system for emulating EIT images using breath sounds comprising:
storage configured to store:
at least one set of recorded breath sounds detected from each of a plurality of patients,
at least one EIT image from each of the plurality of patients where an EIT image corresponds to a set of recorded breath sounds for a given patient in the plurality of patients, and
one or more processors configured to:
generate an intermediate representation of at least one of the at least one set of recorded breath sounds,
encode at least one of the at least one EIT images to generate an EIT embedding corresponding to an associated set of recorded breath sounds, and
map each intermediate representation to the corresponding EIT embedding.

4. The method of claim 1 wherein the at least one set of recorded breath sounds comprises at least one breath cycle.

5. The method of claim 1 wherein the at least one EIT image is a plurality of images.

6. The method of claim 1 wherein the at least one set of recorded breath sounds comprises a plurality of sets recorded from a plurality of transducers.

7. The method of claim 6 wherein the at least one set of recorded breath sounds comprises a plurality of sets recorded from a single transducer.

8. The method of claim 6 wherein the plurality of transducers are analog.

9. The method of claim 6 wherein intermediate representations are mapped against the EIT image embedding.

10. The method of claim 6 wherein the plurality of transducers are integrated into a garment worn by a patient.

11. The method of claim 1 wherein the encoding is performed in a variational auto encoder.

12. The method of claim 1 wherein the encoding is performed in one of a group comprising a Generative Adversarial Network, a Principal Component Analysis, and a Linear Auto Encoder.

13. The method of claim 1 wherein the intermediate representation captures the frequency characteristics of a set of recorded breath sounds.

14. The method of claim 13 wherein conversion of the breath sounds into frequency characteristics is performed by calculating one of a group comprising a spectrogram, a mel spectrogram, and an MFCC.

15. The method of claim 2 wherein a set of recorded breath sounds comprises at least one breath cycle and a breath cycle comprises an inhale portion and an exhale portion.

16. The method of claim 15 further comprising the step of detecting breath cycle boundaries comprising the beginning and ending of the breath cycle.

17. The method of claim 16 wherein the boundary of the breath cycle forms a bounding box for detection of frequency characteristics of the recorded breath songs.

18. The method of claim 16 wherein the step of detecting the breath cycle boundaries [FastRCNN]

19. The system of claim 3 encoding is performed in a group comprising a Variational Auto Encoder, a Generative Adversarial Network, a Principal Component Analysis, and a Linear Auto Encoder.

20. The system of claim 3 further comprising a garment having integrated therein at least one audio transducer for detecting thoracic sounds.
